(19) United States
(12) Patent Application Publication
(10) Pub. No.: US 2025/0288820 A1
(43) Pub. Date: Sep. 18, 2025

(54) REDUCED IMPEDANCE TRANSDUCER ARRAY FOR APPLYING ALTERNATING ELECTRIC FIELDS AND METHODS OF PRODUCTION AND USE THEREOF

(71) Applicant: Novocure GmbH, Baar (CH)

(72) Inventor: Richard Deslauriers, Woodbury, CT (US)

(21) Appl. No.: 19/079,692

(22) Filed: Mar. 14, 2025

Related U.S. Application Data
(60) Provisional application No. 63/565,943, filed on Mar. 15, 2024.
(60) Provisional application No. 63/657,448, filed on Jun. 7, 2024.

(51) Int. Cl. A61N 1/40; A61N 1/04; A61N 1/36

(52) U.S. Cl. CPC A61N 1/40; A61N 1/0476; A61N 1/36002

(57) ABSTRACT

Assemblies, systems, and kits for use in a TTField-generating system are disclosed that include a conductive hydrogel or conductive adhesive in combination with a coating comprising, or formed from, an iodine solution. Also disclosed are methods of producing and using the assemblies, systems, and kits. Further disclosed are methods of applying an iodine solution, a conductive hydrogel or conductive adhesive, and a TTField-generating system to the skin of a subject.

### CROSS REFERENCE TO RELATED APPLICATIONS/INCORPORATION BY REFERENCE STATEMENT

The subject application claims benefit under 35 USC § 119(e) of Provisional Applications U.S. Ser. No. 63/565,943, filed Mar. 15, 2024; and U.S. Ser. No. 63/657,448, filed Jun. 7, 2024. The entire contents of the above-referenced patent applications are hereby expressly incorporated herein by reference.

### BACKGROUND

Tumor Treating Fields (TTFields) are low intensity (e.g., 1-3 V/cm) alternating electric fields within the intermediate frequency range (such as, but not limited to, 50 kHz to 1 MHz, for example, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrode arrays is located to the left and right (LR) of the tumor, and the other pair of electrode arrays is located anterior and posterior (AP) to the tumor. TTFields are approved for the treatment of glioblastoma multiforme (GBM), and may be delivered, for example, via the OPTUNE® system (Novocure GmbH, Baar, Switzerland), which includes transducer arrays placed on the patient's shaved head.

Traditionally, each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces impedance/interference. The device is intended to be continuously worn by the patient for 2-4 days, or parts thereof, before removal for hygienic care and re-shaving (if necessary), followed by reapplication with a new set of arrays. As such, the medical gel remains in substantially continuous contact (for example, 12-24 hours per day) with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

Various types of medical gels are known in the art. One particular type of gel useful as part of a TTField-generating system is a conductive hydrogel. Hydrogels are three-dimensional (3-D) networks of hydrophilic polymers that can swell in water and hold a large amount of water while maintaining their structure due to chemical or physical cross-linking of individual polymer chains. Hydrogels are used in many fields, which include the medical sciences if the hydrogels are nontoxic and compatible with biological environments. (Bahram et al. (2016) “An Introduction to Hydrogels and Some Recent Applications.” Emerging Concepts in Analysis and Applications of Hydrogels. InTech Open).

However, dermatologic adverse events (dAEs) have been observed with the use of existing medical grade hydrogels with TTField generating systems at an incidence rate of 16% and 22% in the phase III trial and the post-marketing surveillance program, respectively; these dAEs include (but are not limited to) allergic and irritant dermatitis, mechanical lesions, ulcers, and skin infections. In particular, irritant contact dermatitis and allergic contact dermatitis can arise from chemical irritation from and allergy to the hydrogel, respectively, while maceration of the skin due to prolonged exposure to the hydrogel can cause lesions/ulcers on the skin, and these lesions/ulcers are subsequently susceptible to infection. These adverse events are exacerbated by the requirement that the hydrogel remain in continuous contact with the patient's skin for lengthy periods of time (for example, 12-24 hours per day) over multiple days at a time without an extended period of “breathability” between application of TTField arrays to the skin. (Lacouture et al. (2014) Seminars in Oncology, 41: S1-S14).

Currently available medical hydrogels typically have pH's that are too acidic for long term wear and thus are damaging to the skin upon extended exposure thereto. In addition, the bottom liner adhesion (e.g., skin adhesion) rate for the currently available hydrogels typically is not high enough to provide the necessary level of attachment to skin for the wear time required. Also, adjustment of either of these two properties can increase the resistivity (impedance) of the hydrogel, thus affecting the ability of the hydrogel to pass electrical current therethrough. In addition, when a hydrogel comes into contact with sweat over the wear period, the hydrogel swells and degrades, which increases resistivity (impedance). Further, loss of the hydrogel interface over the approximately three-day wear period (such as, but not limited to, by erosion of the adhesiveness and conductivity of the hydrogel) reduces the standard current/electric field generated by the TTField system and thus decreases the functionality and overall effectiveness of the TTField treatment.

Because of this extended exposure and the concomitant unique usage of hydrogels with the TTField system, new and improved conductive gel formulations and assemblies containing same are desired that possess multiple properties that are unique and vary from the properties typically possessed by currently available medical grade hydrogels. In particular, there is a need for a reduced impedance conductive gel formulation and for methods to effect a reduced impedance interface between the transducer array and the patient's skin. It is to such assemblies, as well as kits and transducer arrays containing same and methods of producing and using same, that the present disclosure is directed.

### BRIEF DESCRIPTION OF THE DRAWINGS

FIG. 1 is a schematic representation of a general electrode assembly disposed upon a patient's skin, in which a conductive gel (e.g., conductive hydrogel or conductive adhesive) provides an adhesion interface between the electrode and a patient's skin.

FIG. 2 is a representation of two existing transducer arrays for use in a TTField-generating system, wherein the transducer array in the upper panel includes 13 electrode-conductive gel assemblies (showing the skin facing side), and wherein the transducer array in the lower panel includes 20 electrode-conductive gel assemblies and has been disposed upon a patient's skin (showing the outward-facing side).

FIG. 3 is a perspective view of one non-limiting embodiment of an assembly constructed in accordance with the present disclosure, wherein the assembly includes an electrode, a conductive gel (e.g., conductive hydrogel or conductive adhesive), and a coating of an iodine solution or residue thereof.

FIG. 4 is a perspective view of the assembly of FIG. 3 further comprising a liner disposed on the iodine solution coating, conductive gel, and electrode.

FIG. 5 is a schematic representation of another non-limiting embodiment of an assembly constructed in accordance with the present disclosure.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise required by context, singular terms shall include pluralities and plural terms shall include the singular.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The terms “subject” and “patient” as used herein include human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “target region,” as used herein, refers to a region containing all or a portion of the cancer, cancer cells, and/or tumor to be treated.

Turning now to the inventive concept(s), certain non-limiting embodiments thereof include the use of a coating of iodine in combination with a conductive gel (for example, a conductive hydrogel or conductive adhesive) and a transducer array of an alternating electric field-generating system (such as, but not limited to, a TTField-generating system) to reduce impedance of the system and provide less resistive heating, thereby allowing the power of the transducer array to be increased. The iodine coating may be topically applied to the skin of the subject to which the conductive hydrogel (or conductive adhesive) and transducer array are subsequently applied, or the iodine coating may be disposed on a surface of the semi-solid conductive hydrogel (or conductive adhesive). The coating of iodine in combination with a conductive gel (e.g., conductive hydrogel or conductive adhesive) may comprise iodine dispersed throughout at least a portion of the hydrogel (or conductive adhesive), and in certain embodiments, may comprise iodine dispersed throughout the hydrogel (or conductive adhesive). Alternatively, during the methods of use, at least a portion of the iodine may migrate into at least a portion of the hydrogel and become dispersed throughout at least a portion of the hydrogel. In certain embodiments, the iodine may be dispersed homogeneously throughout the hydrogel (or conductive adhesive). In certain embodiments, the iodine may not be dispersed homogeneously throughout the hydrogel (or conductive adhesive). Also, in addition to reducing impedance, the use of iodine in the manner described herein may change the tackiness of the conductive hydrogel/adhesive.

In certain (but non-limiting) embodiments, the conductive hydrogel (or conductive adhesive) may contain a salt dispersed therein, as discussed further below. Such salt-containing conductive hydrogel (or conductive adhesive) may be suitable for use in any of the compositions, assemblies, kits or methods as described herein. Examples of suitable salts are discussed further below, and include iodide salts such as sodium iodide, silver iodide and the like.

Certain non-limiting embodiments of the present disclosure are directed to an assembly that includes a conductive hydrogel (or conductive adhesive) as described herein for placement between at least one electrode and a patient's skin, and a coating comprising, or formed from, an iodine solution. The conductive hydrogel (or conductive adhesive) has a first surface and a second surface, wherein the first surface of the conductive hydrogel (or conductive adhesive) is designed and configured to adhere to at least a portion of a surface of the at least one electrode, and the second surface of the conductive hydrogel (or conductive adhesive) is for application to a patient's skin. The coating comprising or formed from the iodine solution can be in contact with at least a portion of the second surface of the conductive hydrogel (or conductive adhesive). The iodine solution may be applied directly to the second surface of the conductive hydrogel (or conductive adhesive), or the iodine solution may be applied to the subject's skin, and then the second surface of the conductive hydrogel (or conductive adhesive) is adhered thereto. When the iodine solution is applied to the surface of the conductive hydrogel (or conductive adhesive), the assembly may further comprise a liner disposed on the iodine solution (or coating resulting from the iodine solution) and covering at least a portion of the second surface of the conductive hydrogel (or conductive adhesive). In such assemblies, the coating of iodine in combination with a conductive gel (e.g., conductive hydrogel or conductive adhesive) may comprise iodine dispersed throughout at least a portion of the hydrogel (or conductive adhesive). Alternatively, during the methods of use, at least a portion of the iodine may migrate into at least a portion of the hydrogel and become dispersed throughout at least a portion of the hydrogel. For example, the iodine may become dispersed throughout the hydrogel. In any of these embodiments, the assembly may or may not lack homogeneity of iodine migrating into the conductive hydrogel (or conductive adhesive) between the second surface and the first surface.

Any iodine solutions known in the art or otherwise contemplated herein may be utilized in accordance with the present disclosure, so long as the iodine solution is capable of functioning as described herein (i.e., by reducing impedance and providing less resistive heating). Non-limiting examples of iodine solutions that may be utilized in accordance with the present disclosure include povidone-iodine, Iodixanol, an iodophor, elemental iodine, complexed iodine, and combinations thereof. In some embodiments, optionally, the iodine solution may further comprise an iodide salt (e.g., sodium iodide, silver iodide, etc.) or hydrogen iodide.

In certain particular (but non-limiting) embodiments, the iodine solution comprises an iodophor (i.e., a preparation containing iodine that is complexed with a solubilizing agent, such as (but not limited to) a surfactant or water-soluble polymer). Iodophors are formed by mixing iodine with a solubilizing agent, which allows the iodine to become water-soluble. This results in a material that can release free iodine when in solution. Non-limiting examples of solubilizing agents that can be utilized in accordance with the present disclosure include povidone, polyvinylpyrrolidone, polyvinyl alcohol, starch derivatives (e.g., dextrin forming cadexomer iodine), chitosan, cellulose, and the like, as well as combinations thereof.

The conductive hydrogel (or conductive adhesive) may be in any form that allows the assembly to function in accordance with the present disclosure. The conductive hydrogel or conductive adhesive may be polymeric or may be a polymerized conductive hydrogel or conductive adhesive. In certain particular (but non-limiting) embodiments, the conductive hydrogel (or conductive adhesive) is sterile. In addition, in certain non-limiting embodiments, the conductive hydrogel (or conductive adhesive) will not substantially degrade upon exposure to sterilization conditions that include gamma rays or ethylene oxide gas.

In certain particular (but non-limiting) embodiments, the conductive hydrogel (or conductive adhesive) is semi-solid.

The conductive hydrogel may be formed of any hydrophilic polymer that allows the hydrogel to function in accordance with the present disclosure. For example (but not by way of limitation), the hydrogel (or conductive adhesive) may be a polyacrylic acid gel, a povidone gel, or a cellulose gel. In addition, the hydrogel (or conductive adhesive) may comprise at least one of chitosan, alginate, agarose, methylcellulose, hyaluronan, collagen, laminin, matrigel, fibronectin, vitronectin, poly-1-lysine, proteoglycans, fibrin glue, gels made by decellularization of engineered and/or natural tissues, as well as any combinations thereof. Further, the gel may comprise at least one of polyglycolic acid (PGA), polylactic acid (PLA), poly-caprolactone (PCL), polyvinylpyrrolidone (PVP), polyvinyl alcohol (PVA), polyethylene glycol (PEG), methyl methacrylate, poly(methyl methacrylate) (PMMA), poly(2-hydroxyethyl methacrylate) (PolyHEMA), poly(glycerol sebacate), polyurethanes, poly(isopropylacrylamide), poly(N-isopropylacrylamide), or any combination thereof. The polymers of the conductive hydrogel may be provided with any polymer chain length or molecular weight that allows the gel compositions to function as described herein.

The conductive adhesive may be a conductive adhesive composite and can comprise a dielectric material and conductive particles dispersed within the dielectric material. In some embodiments, at least a portion of the conductive particles can define a conductive pathway through a thickness of the conductive adhesive composite. In some aspects, the dielectric material of the electrode assemblies is a polymeric adhesive. Optionally, in these aspects, the polymeric adhesive can be an acrylic adhesive or a silicone adhesive. In some aspects, the conductive particles can comprise carbon. Optionally, in these aspects, the conductive particles can comprise graphite powder. Additionally, or alternatively, the conductive particles can comprise carbon flakes. Additionally, or alternatively, the conductive particles can comprise carbon granules. Additionally, or alternatively, the conductive particles can comprise carbon nanotubes. Additionally, or alternatively, the conductive particles can comprise carbon black powder. Additionally, or alternatively, the conductive particles can comprise carbon microcoils. Additionally, or alternatively, the conductive particles can comprise metal particles. In further aspects, the conductive adhesive composite further comprises a polar material (e.g., a polar salt). The polar salt can be a quaternary ammonium salt, such as a tetra alkyl ammonium salt. Exemplary conductive adhesive composites, as well as methods for making such conductive adhesive composites, are disclosed in U.S. Pat. Nos. 8,673,184 and 9,947,432, which are incorporated herein by reference for all purposes. In exemplary aspects, the conductive adhesive composite can be a dry carbon/salt adhesive, such as the OMNI-WAVE™ adhesive compositions manufactured and sold by FLEXCON® (Spencer, MA, USA); or ARcare® 8006 electrically conductive adhesive composition manufactured and sold by Adhesives Research, Inc. (Glen Rock, PA, USA). In further exemplary aspects, it is contemplated that the conductive adhesive composite can comprise a layer of an electrically conductive adhesive, such as for example, from use (by removal of the transfer film layer) of Electrically Conductive Adhesive Transfer Tape 9712 or Electrically Conductive Adhesive Transfer Tape 9713 (both manufactured by 3M™). For example, the conductive adhesive could be an acrylic adhesive filled with carbon fibers, or the conductive adhesive could be an acrylic adhesive filled with carbon powder.

The conductive hydrogel (or conductive adhesive) may be provided with any pH that does not damage the skin of a patient or cause chemical irritation of the skin upon prolonged exposure to the gel. For example (but not by way of limitation), the gel may have a pH of about 6, about 6.5, about 7, about 7.5, about 8, as well as a range formed from any of the above values (e.g., a range of from about 6 to about 8, a range of from about 6.5 to about 7.5, etc.).

The conductive hydrogel (or conductive adhesive) may be provided with any level of volume resistivity that maximizes the conductiveness of the gel. For example (but not by way of limitation), the gel may have a volume resistivity of less than about 100 Ohm-in, less than about 95 Ohm-in, less than about 90 Ohm-in, less than about 85 Ohm-in, less than about 80 Ohm-in, less than about 75 Ohm-in, less than about 70 Ohm-in, less than about 65 Ohm-in, less than about 60 Ohm-in, less than about 55 Ohm-in, less than about 50 Ohm-in, less than about 45 Ohm-in, less than about 40 Ohm-in, less than about 35 Ohm-in, less than about 30 Ohm-in, less than about 25 Ohm-in, less than about 20 Ohm-in, less than about 15 Ohm-in, less than about 10 Ohm-in, or lower, as well as a range formed of any of the above values (e.g., a range of from about 10 Ohm-in to about 100 Ohm-in, etc.) and a range that combines two integers that fall between two of the above-referenced values (e.g., a range of from about 13 Ohm-in to about 96 Ohm-in, etc.).

The conductive hydrogel (or conductive adhesive) may be provided with any thickness that allows the gel to function in accordance with the present disclosure. Non-limiting examples of thicknesses that may be utilized in accordance with the present disclosure include about 1 μm, about 5 μm, about 10 μm, about 15 μm, about 20 μm, about 25 μm, about 30 μm, about 35 μm, about 40 μm, about 45 μm, about 50 μm, about 75 μm, about 100 μm, about 150 μm, about 200 μm, about 250 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, about 1000 μm, about 1100 μm, about 1200 μm, about 1300 μm, about 1400 μm, about 1500 μm, about 1600 μm, about 1700 μm, about 1800 μm, about 1900 μm, about 2000 μm, about 2100 μm, about 2200 μm, about 2300 μm, about 2400 μm, about 2500 μm, about 2600 μm, about 2700 μm, about 2800 μm, about 2900 μm, about 3000 μm, or higher, as well as a range that combines any two of the above-referenced values (e.g., a range of from about 5 μm to about 3000 μm, a range of from about 10 μm to about 2500 μm, a range of from about 25 μm to about 2500 μm, etc.), and a range that combines two integers that fall between two of the above-referenced values (e.g., a range of from about 12 μm to about 480 μm, etc.).

The conductive hydrogel (or conductive adhesive) may be provided with any skin adhesion rate that allows the gel to function in accordance with the present disclosure. For example (but not by way of limitation), the skin adhesion rate of the gel may be at least about 100 g/inch, at least about 110 g/inch, at least about 120 g/inch, at least about 130 g/inch, at least about 140 g/inch, at least about 150 g/inch, at least about 160 g/inch, at least about 170 g/inch, at least about 180 g/inch, at least about 190 g/inch, at least about 200 g/inch, at least about 210 g/inch, at least about 220 g/inch, at least about 230 g/inch, at least about 240 g/inch, at least about 250 g/inch, at least about 260 g/inch, at least about 270 g/inch, at least about 280 g/inch, at least about 290 g/inch, at least about 300 g/inch, or higher, as well as a range of any of the above values (a range of from about 120 g/inch to about 300 g/inch, etc.), and a range that combines two integers that fall between two of the above-referenced values (e.g., a range of from about 115 g/inch to about 295 g/inch, etc.).

In certain non-limiting embodiments, the conductive hydrogel (or conductive adhesive) has at least one of a decreased polymer chain length and an added free salt when compared to existing gel compositions; the decrease in polymer chain length and increase in free salt concentration maximizes the conductivity of the gel while reducing the occurrence of skin irritation caused by the gel. In a particular (but non-limiting) embodiment, the conductive hydrogel (or conductive adhesive) comprises a free salt present via incorporation within the gel or as one layer of a multi-layered gel (e.g., a bilayered gel). The term “free salt” refers to salt molecules that are not incorporated as part of the polymerized chain structure but rather are floating substantially freely within the gel and thus are a source of free ions that conduct electricity and thus reduce impedance.

When present, the free salt present in the gel may be any salt or other substance that serves as a source of free ions that are capable of floating substantially freely within the gel, wherein the free ions serve to conduct electricity and thus reduce impedance. In certain particular (but non-limiting) embodiments, the free salt present in the gel is a source of chloride ions, citrate ions, silver ions, iodide ions, etc., or any other ions that are known to be good conductors. Non-limiting examples of free salts that may be utilized in accordance with the present disclosure are salts that contain potassium (K), ammonium (NH₄⁺), sodium (Na), nitrate, bicarbonate, and the like. Particular non-limiting examples of free salts that may be utilized in accordance with the present disclosure are NaCl, KCl, CaCl₂, MgCl₂, ZnCl₂, silver iodide (AgI), sodium iodide (NaI), silver dihydrogen citrate (SDC), sodium dihydrogen citrate, combinations thereof, and the like.

When present, the free salt present in the gel may be provided with any concentration that allows the gel compositions to function as described herein. For example (but not by way of limitation), the free salt concentration may be at least about 0.1 mM, about 0.5 mM, about 1 mM, about 2 mM, about 3 mM, about 4 mM, about 5 mM, about 6 mM, about 7 mM, about 8 mM, about 9 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1 M, or higher, as well as any range that combines any two of the above-referenced values (e.g., a range of from about 0.1 mM to about 100 mM, a range of from about 1 mM to about 50 mM, etc.), and a range that combines two integers that fall between two of the above-referenced values (e.g., a range of from about 12 mM to about 550 mM, etc.).

In other non-limiting embodiments, the free salt concentration, when present, is dependent upon the frequency(ies) of the alternating electric field. For example (but not by way of limitation), the range of the free salt concentration may be based upon a range of frequencies of the alternating electric field. Non-limiting examples include a range of from about 0.1 mM to about 50 mM when the alternating electric field has a frequency in a range of from about 50 kHz to about 150 kHz, a range of from about 50 mM to about 100 mM when the alternating electric field has a frequency in a range of from about 150 kHz to about 300 kHz, etc.

Alternatively and/or in addition thereto, in a particular non-limiting embodiment, the conductive hydrogel (or conductive adhesive) comprises one or more of the following chemical and structural features/properties: a free salt present at a concentration in a range of from about 0.1 mM to about 1 M; a pH in a range of from about 6 to about 8; a volume resistivity of less than about 100 Ohm-in; and a skin adhesion rate of at least about 100 g/inch.

In addition, given the prolonged exposure of the gel composition to the patient's skin, the gel should be optimized for use at body temperature (e.g., in a range of from about 34° C. to about 40° C.).

In certain particular (but non-limiting) embodiments, the conductive gel (e.g., conductive hydrogel or conductive adhesive) is a multi-layer structure that comprises: a scrim having a first and a second side with a gel tie layer attached to the first side of the scrim, and a gel skin layer attached to the second side of the scrim. The gel tie layer is designed for contact with the transducer array, while the gel skin layer is designed for contact with the patient's skin. The gel tie and gel skin layers are both formed of any of the conductive gels described or otherwise contemplated herein, and may be formed of the same or different conductive gels. The scrim may be formed of any material that allows the composition to function in accordance with the present disclosure; in particular, the material from which the scrim is formed is typically selected to optimize conductivity and minimize resistance of the composition. A non-limiting example of a material from which the scrim can be formed is spun nylon or polyester. In certain non-limiting embodiments, the gel comprises and/or consists of at least one perforation extending through the entirety of the multi-layer structure. In certain non-limiting embodiments, the gel comprises at least one male protrusion(s) extending from a side of the gel skin layer that engages a patient's skin.

The assembly may optionally further include a liner that is disposed on the iodine solution/coating and that covers at least a portion of the second surface of the conductive hydrogel (or conductive adhesive) to maintain the iodine solution/coating in place on the conductive hydrogel (or conductive adhesive) and/or to maintain the sterility of the conductive hydrogel/adhesive until use.

In certain particular (but non-limiting) embodiments, the assembly further comprises at least one electrode, wherein the first surface of the conductive hydrogel (or conductive adhesive) is adhered to at least a portion of the surface of the at least one electrode.

Any type of conductive or non-conductive electrode(s) and/or transducer array(s) that can be utilized for generating an alternating electric field that are known in the art or otherwise contemplated herein may be utilized for the generation of the alternating electric field in accordance with the present disclosure. Non-limiting examples of electrodes and transducer arrays that can be utilized for generating an alternating electric field in accordance with the present disclosure include those that function as part of a TTFields system as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016. For example, in certain non-limiting embodiments, the electrode may be an insulated electrode that comprises at least one conducting layer, and a high capacitance layer having an upper surface and a lower surface; in addition, at least one opening may be disposed between the upper surface and the lower surface of the high capacitance layer. When this electrode structure is utilized, the first surface of the conductive hydrogel (or conductive adhesive) may be adhered to the lower surface of the high capacitance layer of the electrode.

Alternatively (and/or in addition thereto), the at least one electrode of the assemblies may include a ceramic dielectric material or a non-ceramic dielectric material positioned over a flat conductor. Examples of the latter include polymer films disposed over pads on a printed circuit board or over flat pieces of metal. Other non-limiting examples of electrodes that can be utilized in accordance with the present disclosure include electrodes that are not capacitively coupled; in this situation, the at least one electrode is implemented using a region of a conductive material that is configured for placement against a person's body, with no insulating dielectric layer disposed between the conductive element and the body. Examples of the conductive material include, but are not limited to, a conductive film, a conductive fabric, and/or a conductive foam. Other alternative constructions of electrodes may be utilized in accordance with the present disclosure, as long as they are capable of delivering TTFields to the person's body as described herein.

Certain non-limiting embodiments of the present disclosure are related to transducer arrays that include one or more of any of the assemblies disclosed or otherwise contemplated herein. For example (but not by way of limitation), the transducer array may include at least about one assembly, at least about two assemblies, at least about three assemblies, at least about four assemblies, at least about five assemblies, at least about six assemblies, at least about seven assemblies, at least about eight assemblies, at least about nine assemblies, at least about 10 assemblies, at least about 11 assemblies, at least about 12 assemblies, at least about 13 assemblies, at least about 14 assemblies, at least about 15 assemblies, at least about 16 assemblies, at least about 17 assemblies, at least about 18 assemblies, at least about 19 assemblies, at least about 20 assemblies, at least about 21 assemblies, at least about 22 assemblies, at least about 23 assemblies, at least about 24 assemblies, at least about 25 assemblies, at least about 26 assemblies, at least about 27 assemblies, at least about 28 assemblies, at least about 29 assemblies, at least about 30 assemblies, at least about 35 assemblies, at least about 40 assemblies, at least about 45 assemblies, at least about 50 assemblies, at least about 55 assemblies, at least about 60 assemblies, at least about 65 assemblies, at least about 70 assemblies, at least about 75 assemblies, at least about 80 assemblies, at least about 85 assemblies, at least about 90 assemblies, at least about 95 assemblies, at least about 100 assemblies, or more, as well as a range of any of the above values (e.g., a range of from about 2 assemblies to about 30 assemblies, a range of from about 9 assemblies to about 20 assemblies, etc.), or a range formed of any two values that fall between two of the above values.

In certain particular (but non-limiting) embodiments, pairs of transducer arrays cooperate in a TTField-generating system to generate an alternating electric field having a frequency in a range of from about 50 kHz to about 1 MHz (for example, but not by way of limitation, 50-500 kHz) upon application to a patient's skin in combination with the conductive hydrogels or conductive adhesives and iodine solutions/coatings. Examples of transducer arrays that function as part of a TTField system are known in the art and are described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016. Therefore, no further description thereof is deemed necessary.

Certain non-limiting embodiments of the present disclosure are directed to a method that comprises applying any of the assemblies (containing conductive hydrogel/conductive adhesive and iodine solution/coating) disclosed or otherwise contemplated herein to a skin of a patient, applying at least one electrode to a first surface of the conductive hydrogel (or conductive adhesive) applied to the skin of the patient, and generating an alternating electric field for a period of time. If the assembly does not contain the iodine coating/solution, then the iodine coating/solution is applied to the skin of the patient prior to application of the gel/adhesive and electrode.

Certain non-limiting embodiments of the present disclosure are directed to a method that comprises applying two or more of any of the transducer arrays disclosed or otherwise contemplated herein to a skin of a patient and generating an alternating electric field for a period of time. If the transducer array does not contain the iodine coating/solution, then the iodine coating/solution is applied to the skin of the patient prior to application of the hydrogel/transducer array.

Certain non-limiting embodiments of the present disclosure are directed to a method that includes the steps of: applying any of the iodine coatings/solutions disclosed or otherwise contemplated herein to a portion of a skin of a patient; applying or contacting any of the conductive hydrogels (or conductive adhesives) disclosed or otherwise contemplated herein to at least a portion of the skin to which the iodine coating/solution has been applied; applying or contacting at least one of any of the transducer arrays for a TTField-generating system disclosed or otherwise contemplated herein to at least a portion of the skin to which the iodine coating/solution and conductive hydrogel (or conductive adhesive) have been applied; and generating an alternating electric field for a period of time.

Certain additional non-limiting embodiments of the present disclosure further include a method that includes the steps of: applying any of the iodine solutions/coatings disclosed or otherwise contemplated herein to a portion of a skin of a patient; including any of the conductive hydrogels/adhesives disclosed or otherwise contemplated herein as a component of at least one of any of the transducer arrays for a TTField-generating system disclosed or otherwise contemplated herein (or applying or contacting the conductive hydrogel/adhesive to at least a portion of the at least one transducer array for a TTField-generating system); applying or contacting the at least one transducer array for the TTField-generating system to at least a portion of the skin to which the iodine solution/coating has been applied, thereby contacting the at least one transducer array to the skin of the patient via the conductive hydrogel/adhesive; and generating an alternating electric field for a period of time.

In certain particular (but non-limiting) embodiments of the various methods disclosed herein, the electrode generates an alternating electric field within a target region of the patient. The target region typically comprises at least one tumor (or resection cavity after removal of at least one tumor), and the generation of the alternating electric field selectively destroys or inhibits growth of the tumor (or residual cancer cells after removal of the at least one tumor). The alternating electric field may be generated at any frequency that selectively destroys or inhibits growth of the tumor (or cancer cells). For example (but not by way of limitation), the alternating electric field may have a frequency of about 50 kHz, about 75 kHz, about 100 kHz, about 125 kHz, about 150 kHz, about 175 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the target region/subject/cancer cells, so long as the alternating electric field is capable of functioning in accordance with the present disclosure. For example (but not by way of limitation), the alternating electric field may have a field strength in the target region/subject/cancer cells of at least about 1 V/cm, about 1.5 V/cm, about 2 V/cm, about 2.5 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two values that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.). Generally, it is desired to utilize the highest field strength possible without causing overheating, with field intensity typically being capped by temperature measurements.

In some instances, the electric field in at least a portion of the target region/subject/cancer cells is induced by an applied voltage of at least 50 V RMS, and optionally, the applied voltage is at least 100 V RMS. In some embodiments, an applied voltage of at least 50 V induces an alternating electric field with a field strength of at least 1 V/cm (e.g., at least 5 V/cm) in at least a portion of the target region/subject/cancer cells.

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure GmbH, Baar, Switzerland)) operate in two directions in order to increase the chances that a dividing cell will be aligned with the electric field such that the electric field can have the desired anti-mitotic effect. The two directions may or may not be perpendicular. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session (e.g., continuous application) as well as multiple sessions with minor breaks in between sessions (e.g., consecutive application for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, or longer, as well as a range formed from any of the above values (e.g., a range of from about 1 hour to about 6 months, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 cumulative hours within 48 consecutive hours. In another particular (but non-limiting) embodiment, the period of time that the alternating electric field is applied is at least about 24 hours, with the device positioned on the body and operational for at least about 80% of that period.

Certain non-limiting embodiments of the present disclosure are related to kits that include any of the components of the TTField generating systems and/or any of the assemblies disclosed or otherwise contemplated herein. For example (but not by way of limitation), the kit may include any of the conductive hydrogels (or conductive adhesives) disclosed or otherwise contemplated herein along with any of the iodine solutions/coatings disclosed or otherwise contemplated herein. In certain embodiments, the conductive hydrogels (or conductive adhesives) may be provided in a form that includes a salt dispersed therein, such as, for example, an iodide salt (e.g., sodium iodide, silver iodide, etc.) as described previously herein, and, optionally, the salt-containing conductive hydrogel (or conductive adhesive) may be provided along with any of the iodine solutions/coatings disclosed or otherwise contemplated herein. Alternatively (and/or in addition thereto), the kit may contain one or more assemblies that includes at least one electrode in combination with the conductive hydrogel(s) (or conductive adhesive(s)) and the iodine-containing solutions/coatings. In yet another alternative (and/or in addition thereto), the kit may contain at least one pair of any of the transducer arrays disclosed or otherwise contemplated herein.

In a particular (but non-limiting) embodiment, the kit comprises any of the conductive hydrogels (or conductive adhesive(s)) disclosed or otherwise contemplated herein for application to a patient's skin and for placement between the patient's skin and at least one transducer array that generates an alternating electric field, wherein the conductive hydrogel (or conductive adhesive) comprises a first surface and a second surface; and any of the iodine coatings/solutions disclosed or otherwise contemplated herein for disposal on at least a portion of the second surface of the hydrogel (or conductive adhesive) or for disposal on at least a portion of the patient's skin.

In a particular (but non-limiting) embodiment, the kit may further include instructions for applying at least a portion of the iodine solution/coating to at least a portion of the conductive hydrogel (or conductive adhesive) and/or to at least a portion of a patient's skin, and/or for applying the conductive hydrogel (or conductive adhesive) (with or without the iodine coating thereon) to a patient's skin, and/or for applying the assembly to a patient's skin.

In certain non-limiting embodiments, the conductive hydrogel (or conductive adhesive) is for application to a patient's skin and for placement between the patient's skin and at least one transducer array that generates an alternating electric field having a frequency in a range of from about 50 kHz to about 1 MHz; in addition, the conductive hydrogel (or conductive adhesive) comprises a first surface and a second surface. Also in this embodiment, the iodine solution/coating is for disposal on at least a portion of the second surface of the conductive hydrogel (or conductive adhesive) and/or for disposal on the patient's skin.

In certain non-limiting embodiments, the kit includes at least one dermatological therapeutic agent. Any type of dermatological therapeutic agent known in the art that would be useful for use in combination with the conductive hydrogels (or conductive adhesives) and a transducer array/TTField-generating system may be utilized in accordance with the present disclosure. Non-limiting examples of dermatological therapeutic agents that may be utilized in accordance with the present disclosure include an anti-microbial agent, an antibiotic, an anti-viral agent, an anti-fungal agent, an anti-inflammatory agent, an anesthetic, an emollient, a cleansing agent, an astringent, and combinations thereof. Further, any type of dermatological therapeutic agent known in the art that may enhance the hydrogel/skin interface, enhance skin conductivity, enhance adhesiveness, and/or reduce or prevent the occurrence of dAEs may be utilized in accordance with the present disclosure including the dermatological therapeutic agents listed above.

In addition, the kit may further include at least one removable liner attached to at least a portion of a surface of the conductive hydrogel (or conductive adhesive) (with or without the iodine coating disposed thereon) for protecting the surface until use. For example (but not by way of limitation), the conductive hydrogel (or conductive adhesive) may have a removable top liner and/or a removable bottom liner attached to the first and second surfaces thereof, respectively.

In certain particular (but non-limiting) embodiments, the kit may further include at least one pair of transducer arrays that generates an alternating electric field having a frequency in a range of from about 50 kHz to about 1 MHz (e.g., from about 50-500 kHz) upon application to a patient's skin in combination with the gel composition (e.g., transducer arrays that function as part of a TTField-generating system). For example (but not by way of limitation), the kit may include at least one pair of transducer arrays, at least about two pairs of transducer arrays, at least about four pairs of transducer arrays, or more, as well as a range of pairs of transducer arrays such as a range of from about one pair of transducer arrays to about 50 pairs of transducer arrays, a range of from about two pairs of transducer arrays to about 20 pairs of transducer arrays, etc.), or a range that combines two integers that fall between a range of from about one pair of transducer arrays to about 50 pairs of transducer arrays.

Examples of transducer arrays that function as part of a TTField system are known in the art and are described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016. Therefore, no further description thereof is deemed necessary.

The conductive hydrogel (or conductive adhesive) may be present in the kit in any form that allows the kit to perform in accordance with the present disclosure. For example, but not by way of limitation, the conductive hydrogel (or conductive adhesive) may be provided in the form of one or more sheets or one or more rolls. In addition, the conductive hydrogel (or conductive adhesive) may be provided within the kit in a single, individual unit/amount, or multiple units/amounts of the conductive hydrogel (or conductive adhesive).

Likewise, the iodine solution/coating may be present in the kit in any form that allows the kit to perform in accordance with the present disclosure. For example, but not by way of limitation, the iodine solution/coating may be provided in a tube or bottle/container or in single use aliquots/ampules, or as a film/coating separate from or attached to the hydrogel.

When one or more transducer arrays are present in the kit, two or more components of the kit may be assembled for use thereof. For example (but not by way of limitation), the conductive hydrogel (or conductive adhesive) may be disposed upon the surface of the electrodes of the transducer arrays and this combination sealed together (either with or without a removable liner disposed upon the conductive hydrogel (or conductive adhesive)) prior to placement of the combination within the kit (which would also contain a separate container(s) of iodine solution/coating). In another example, both the conductive hydrogel (or conductive adhesive) and the iodine solution/coating may be disposed upon the surface of the electrodes of the transducer arrays and this entire combination sealed together (either with or without a removable liner disposed upon the conductive hydrogels (or conductive adhesives)) prior to placement of the combination within the kit.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to applying the iodine coating, hydrogel composition, and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format and/or area/organ to be treated, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the individual components and/or one or more assemblies are disposed. In certain particular (but non-limiting) embodiments, the sealed packaging is substantially impermeable to air (so as to allow the hydrogels/adhesives to substantially maintain their water content) and/or substantially impermeable to light (as certain components may be light sensitive).

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel/adhesive compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with any of the hydrogel/adhesive compositions and/or iodine solutions/coatings disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

In certain particular (but non-limiting) embodiments, the electric field is generated within a target region of the patient. The target region typically comprises at least one tumor (or resection cavity), and the generation of the alternating electric field selectively destroys or inhibits growth of the tumor (or residual cancer cells). The alternating electric field(s) may be generated at any frequency (or frequencies) that selectively destroys or inhibits growth of the tumor (or cancer cells), such as, for example (but not by way of limitation), at any of the frequencies disclosed earlier herein, or within a range of frequencies as disclosed earlier herein.

In certain particular (but non-limiting) embodiments, the assembly/transducer array is maintained upon the patient's skin for at least about three days, which may be a continuous period of time or may comprise non-continuous consecutive periods of time.

In certain particular (but non-limiting) embodiments, the methods disclosed herein may include the steps of: removing the assembly/transducer array from the patient's skin; preparing the patient's skin for another treatment (such as, but not limited to, cleansing of the skin and shaving of the skin, if necessary); and repeating the steps of applying the iodine coating, hydrogel/adhesive, and electrodes/transducer arrays. In addition, this cycle of steps can be repeated as many times as necessary.

When one or more application steps are repeated, the electrodes (in combination with the hydrogel/adhesive and/or iodine coating) may be placed in different positions than their original placement; relocation of the arrays in this manner further minimizes any dAEs that may occur.

One of the many advantages to the use of the iodine coating as disclosed herein is a reduction in the skin's susceptibility to infection. This feature can be further enhanced by the addition of one or more dermatological therapeutic agents to the system, as described elsewhere herein.

As such, any of the kits, assemblies, and/or methods disclosed herein may further comprise at least one dermatological therapeutic agent. Further, any type of dermatological therapeutic agent known in the art that may enhance the hydrogel/skin interface, enhance skin conductivity, enhance adhesiveness, and/or reduce or prevent the occurrence of dAEs may be utilized in accordance with the present disclosure, including any of the dermatological therapeutic agents listed earlier herein.

Certain non-limiting embodiments of the present disclosure are directed to systems that comprise a coating of any of the iodine solutions disclosed or otherwise contemplated herein applied to a skin of a patient, any of the conductive hydrogels/conductive adhesives disclosed or otherwise contemplated herein applied to the iodine-coated skin, and any of the electrodes/transducer arrays disclosed or otherwise contemplated herein applied to the conductive hydrogel/conductive adhesive. The iodine solution/coating may be disposed directly on the skin of the patient before the conductive hydrogel/conductive adhesive and the electrode/transducer array are applied to the skin; alternatively, the iodine solution/coating may be applied to the conductive hydrogel/conductive adhesive (either alone or as part of an assembly that includes the electrode/transducer array) prior to placement of the conductive hydrogel/conductive adhesive on the skin of the patient. As such, the system can be produced by any of the methods disclosed or otherwise contemplated herein.

When the iodine solution/coating is applied directly to the hydrogel/conductive adhesive prior to placement of the conductive hydrogel/conductive adhesive on the skin of the patient, the iodine may remain within the coating on the surface of the conductive hydrogel/adhesive.

Alternatively, at least a portion of the iodine may migrate out of the coating and into at least a portion of the conductive hydrogel/adhesive, whereby the conductive hydrogel/adhesive comprises iodine dispersed throughout at least a portion of the hydrogel (or conductive adhesive).

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

Referring now to the Figures, shown therein are non-limiting embodiments of various components and configurations of the assemblies disclosed herein, along with transducer arrays and TTField-generating systems containing same. While hydrogels are specifically shown in the Figures, a person having ordinary skill in the art should readily understand that the various configurations disclosed herein may be utilized with any conductive gel/adhesive commonly known in the art.

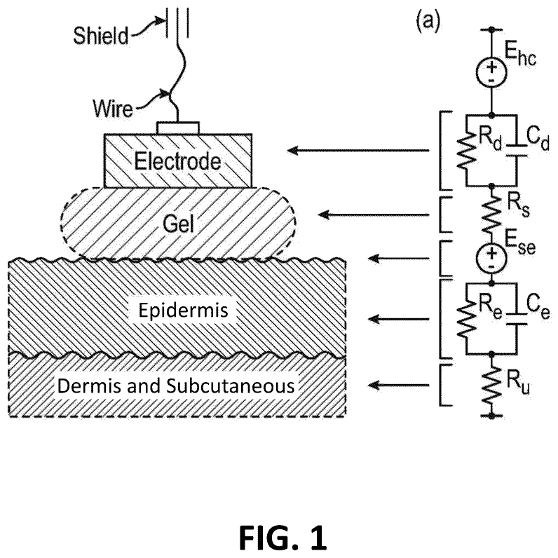

FIG. 1 is a schematic representation of a portion of an existing TTField-generating system that can be utilized in accordance with the present disclosure. As generally shown in FIG. 1, a conductive gel (e.g., conductive hydrogel or conductive adhesive) provides an adhesion interface between at least one insulated electrode of the TTField-generating system and a patient's epidermal layer for delivery of at least one TTField to and/or through the patient's epidermal and/or dermal/subcutaneous layers.

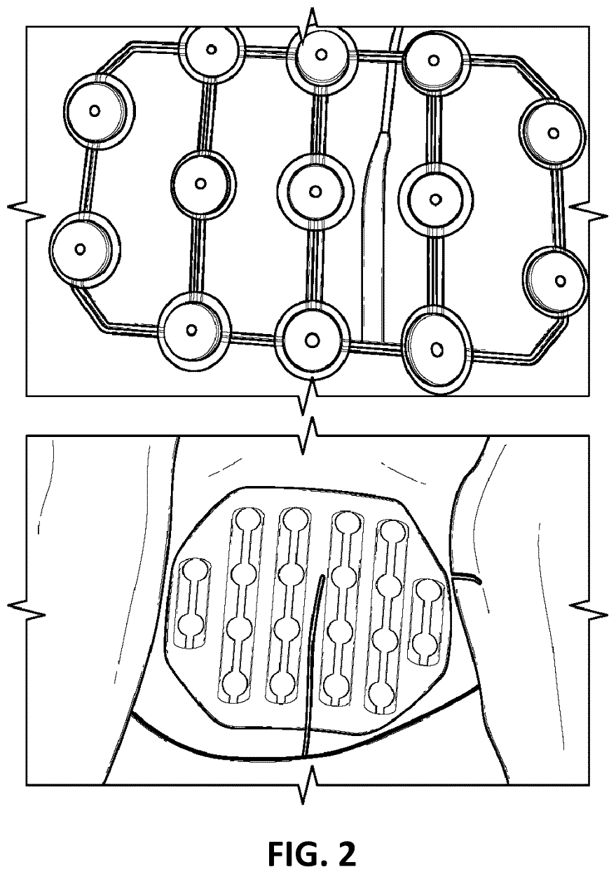

FIG. 2 depicts two existing transducer arrays for use in a TTField-generating system. As can be seen, each of these arrays includes a plurality of the electrode-gel assemblies shown in FIG. 1. The array in the upper panel contains 13 electrodes (showing the skin-facing side), while the array in the lower panel contains 20 electrodes; in addition, the lower panel depicts attachment of the array to a patient's skin (showing the outward-facing side).

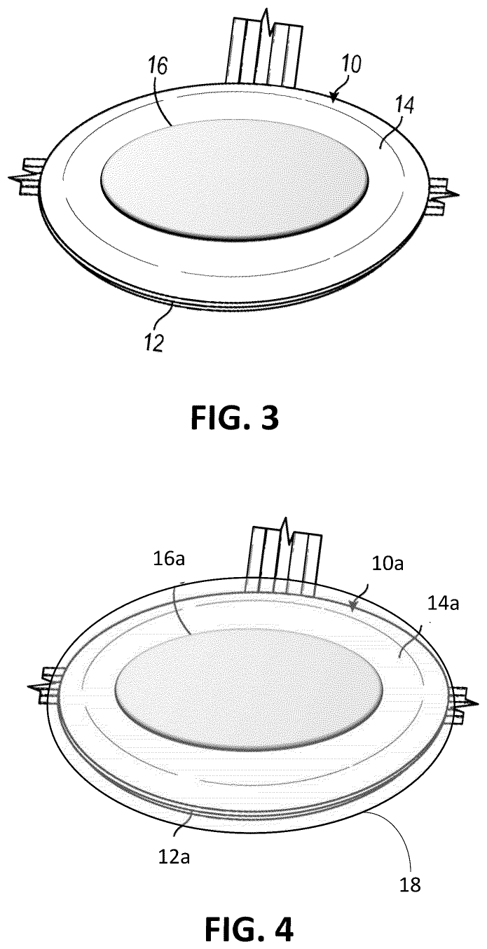

FIG. 3 illustrates an assembly 10 constructed in accordance with the present disclosure. The assembly 10 includes an electrode 12 having a conductive hydrogel layer 14 disposed thereon. Then a coating of an iodine solution 16 is disposed upon the conductive hydrogel layer 14.

The amount of iodine coating 16 deposited on the conductive hydrogel layer 14 may vary, and may simply be sufficient to cover only a portion of the surface of the conductive hydrogel layer 14, or to cover all of the surface of the conductive hydrogel layer 14; alternatively, the volume of iodine coating 16 may be substantial enough to extend throughout and beyond the conductive hydrogel layer 14 when the assembly 10 is pressed onto the surface of a patient's skin.

When the iodine coating 16 is deposited on the conductive hydrogel layer 14, the iodine in the coating 16 may remain on the surface of the conductive hydrogel layer 14. Alternatively, at least a portion of the iodine may migrate out of the coating 16 and into at least a portion of the conductive hydrogel layer 14 so that at least a portion of the conductive hydrogel layer 14 comprises iodine dispersed through at least a portion of the conductive hydrogel layer 14. In certain embodiments, the iodine may be dispersed throughout the conductive hydrogel layer 14.

As shown in FIG. 3, the iodine coating 16 may simply be deposited upon an existing surface of the conductive hydrogel layer 14, and this may be accomplished by any manner known in the art.

The electrodes utilized as part of the assemblies and transducers described herein (such as, but not limited to, the electrode 12) may be constructed in any manner and contain any components necessary to allow the assemblies and transducer arrays to function in accordance with the present disclosure. Certain non-limiting structural embodiments of electrodes are described herein after; however, these structures are provided for illustrative, exemplary purposes only, and should not be construed as limiting to the electrode structure of any of the assemblies of the present disclosure.

FIG. 4 illustrates an assembly 10a that is identical to the assembly 10 of FIG. 3, except that the assembly 10a optionally further includes a liner 18 that is disposed on the iodine solution/coating 16a and that covers at least a portion of a second surface of the conductive hydrogel 14a to maintain the iodine solution/coating 16a in place on the conductive hydrogel 14a and/or to maintain the sterility thereof until use.

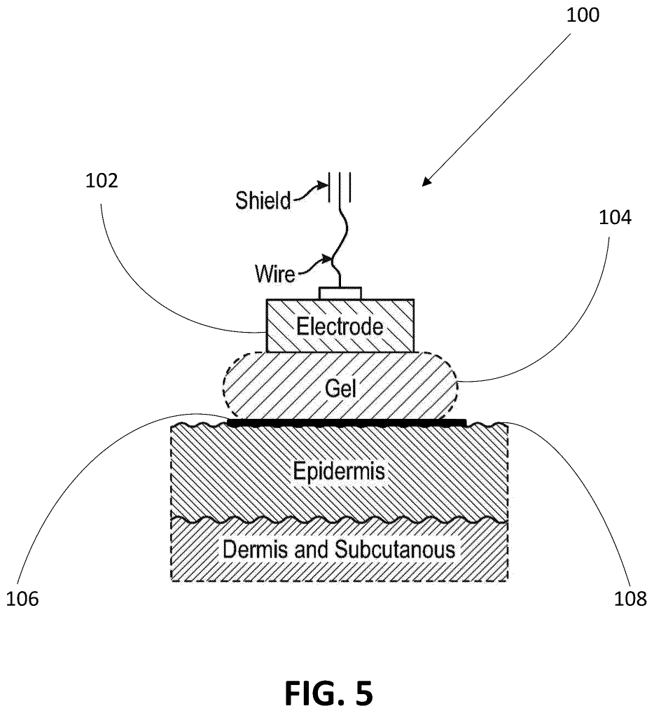

FIG. 5 illustrates a system 100 that comprises an electrode 102, a conductive hydrogel layer 104, and a coating of an iodine solution 106 disposed upon a skin 108 of a patient. The iodine solution/coating 106 may be disposed directly on the skin 108 of the patient before the conductive hydrogel layer 104 and the electrode 102 are applied to the skin 108; alternatively, the iodine solution/coating 106 may be applied to the conductive hydrogel layer 104 (either alone or as part of an assembly that includes the electrode 102 (such as, but not limited to, the assembly 10 of FIG. 3)) prior to placement of the conductive hydrogel layer 104 on the skin 108 of the patient. Therefore, the complete system 100 shown in FIG. 5 can be produced by any of the methods disclosed or otherwise contemplated herein.

When the iodine solution/coating 106 is directly deposited on the conductive hydrogel layer 104 prior to placement of the conductive hydrogel layer 104 on the skin 108 of the patient, the iodine in the coating 106 may remain on the surface of the conductive hydrogel layer 104 during use of the system 100 to produce TTFields. Alternatively, prior to and/or during use of the system 100, at least a portion of the iodine may migrate out of the solution/coating 106 into at least a portion of the conductive hydrogel layer 104 so that the conductive hydrogel layer 104 comprises iodine dispersed through at least a portion of the conductive hydrogel layer 104 during at least a portion of the use of the system 100. In certain embodiments, the iodine may be dispersed throughout the conductive hydrogel layer 14.

### Example 2

Iodine is known for its antibacterial, antiseptic, and disinfectant properties. In addition to the use of tinctures of iodine, povidone-iodine (a combination of molecular iodine and polyvinylpyrrolidone) is widely used due to its broad spectrum of antimicrobial activity and its efficacy against resistant microorganisms such as methicillin-resistant Staphylococcus aureus (MRSA). Povidone-iodine is known to be safe for use on patients of all ages. Povidone-iodine preparations are widely commercially available in the form of single-use swabs and applicators.

Free iodine typically exists as a diatomic molecule (I₂). lodine itself is not a metal and does not ionize in the same way as metals do (i.e., losing electrons to form positive ions). Instead, iodine tends to form negative ions or participate in covalent bonding.

The present disclosure has surprisingly found that use of an iodine solution/coating disposed between the patient's skin and the conductive hydrogel (or conductive adhesive) of a transducer array of an alternating electric field-generating system serves to reduce impedance of the system and provide less resistive heating, thereby allowing the power of the transducer array to be increased.

In an experiment, two 1-inch washers were spaced 6 inches apart on two different hydrogel formulations, both of which had a thickness in a range of from about 0.75 mm inches to about 1.25 mm and contained a nylon scrim. The washers were connected to a Tenma 72-960 LCR meter and delivered a 1 KHz AC signal. A hydrogel containing 0.05% silver nitrate had an average impedance of 19.4 KOhms, and separately a hydrogel containing 3.5% potassium chloride or a hydrogel containing 2.5% sodium chloride had a similar impedance (within 10% of the impedance of the silver nitrate-containing hydrogel). However, a hydrogel containing 5.0% sodium iodide (NaI) had an average impedance of 1.46 KOhms, thereby demonstrating that the presence of NaI reduced impedance by over 13-fold.

In another bench test, the OPTUNE® device (Novocure GmbH, Baar, Switzerland) was utilized at maximum power (200 KHz in this test) for eight minutes with a 7.5% NaCl hydrogel with or without povidone-iodine (one swab per array) being applied to the skin of a human subject prior to application of the hydrogel and the device. The povidone-iodine was swabbed on the skin as a pre-wash and allowed to dry before the hydrogel was applied to the washed surface. The results are shown in Table 1.

TABLE 1

| | |
|---|---|
| Without Iodine | With Iodine |

| | | | | |
|---|---|---|---|---|
| Output | 2005 | mAmps | 2022 | mAmps |
| Impedance | 54 | Ohms | 45 | Ohms |
| Voltage | 110 | Volts | 92 | Volts |

The use of iodine solution/coating (whether applied directly to the skin or applied to the skin-facing surface of the hydrogel) reduces the impedance of the system. The reduction in impedance caused by the use of the iodine solution/coating allows for an increase in the effective surface area of the hydrogel and therefore an increase in the amount of current that can be delivered to the patient.

While not wishing to be bound by any theory, it is possible that the iodine applied to the skin (whether directly applied to the skin or first disposed on the hydrogel and then applied to the skin as part of an assembly) binds to the hair follicles, pores, and/or skin and increases the conductivity of the hair follicles, pores, and/or skin, thereby reducing impedance. Therefore, the use of an iodine solution/coating applied to the skin overcomes impedance issues encountered when the assembly is applied to dead, keratinized skin.

### NON-LIMITING ILLUSTRATIVE EMBODIMENTS OF THE INVENTIVE CONCEPT(S)

Illustrative embodiment 1A. A method, comprising the steps of: (a) applying or contacting an iodine solution to a portion of a skin of a patient; (b) applying or contacting a conductive hydrogel (or conductive adhesive) to at least a portion of the skin to which the iodine solution has been applied; (c) applying or contacting at least one transducer array for a TTField-generating system to at least a portion of the skin to which the iodine solution and conductive hydrogel (or conductive adhesive) have been applied; and (d) generating an alternating electric field for a period of time.

Illustrative embodiment 1B. A method, comprising the steps of: (i) applying or contacting an iodine solution to a portion of a skin of a patient; (ii) including a conductive hydrogel or a conductive adhesive as a component of at least one transducer array for a TTField-generating system, or applying or contacting a conductive hydrogel or a conductive adhesive to at least a portion of the at least one transducer array for a TTField-generating system; (iii) applying or contacting the at least one transducer array for the TTField-generating system to at least a portion of the skin to which the iodine solution has been applied thereby contacting the at least one transducer array to the skin of the patient via the conductive hydrogel or conductive adhesive; and (iv) generating an alternating electric field for a period of time.

Illustrative embodiment 2. The method of Illustrative embodiment 1A or 1B, wherein at least one of: the alternating electric field is applied at a frequency in a range of from about 50 kHz to about 1 MHz; the alternating electric field has a field strength of at least about 1 V/cm in at least a portion of a target region of the subject; the alternating electric field is induced by an applied voltage of at least 50 V RMS; and the period of time that the alternating electric field is applied is at least about 50% of at least about a 24 consecutive hour time period.

Illustrative embodiment 3. The method of any of Illustrative embodiments 1A, 1B, or 2, wherein the iodine solution is selected from the group consisting of povidone-iodine, Iodixanol, an iodophor, elemental iodine, complexed iodine, and combinations thereof; optionally, wherein the iodine solution includes an iodide salt (e.g., sodium iodide, silver iodide, etc.) or hydrogen iodide.

Illustrative embodiment 3A. The method of any of Illustrative embodiments 1-3, wherein the iodine solution comprises an iodophor, and wherein the iodophor comprises iodine complexed with at least one solubilizing agent.

Illustrative embodiment 3B. The method of Illustrative embodiment 3A, wherein the solubilizing agent is selected from the group consisting of povidone, polyvinylpyrrolidone, polyvinyl alcohol, starch derivatives, chitosan, cellulose, and combinations thereof.

Illustrative embodiment 4. The method of any of Illustrative embodiments 1-3B, wherein the conductive hydrogel (or conductive adhesive) has a first surface and a second surface, wherein the first surface of the conductive hydrogel (or conductive adhesive) adheres to at least a portion of a surface of the transducer array, and the second surface of the conductive hydrogel (or conductive adhesive) is applied to the skin to which the iodine solution has been applied.

Illustrative embodiment 5. The method of any one of Illustrative embodiments 1-4, wherein the conductive hydrogel (or conductive adhesive) is a semi-solid conductive hydrogel (or conductive adhesive) having a thickness in a range of from about 25 μm to about 2500 μm, and/or a range of from about 1 mil to about 100 mil.

Illustrative embodiment 5A. The method of any one of illustrative embodiments 1-5, wherein at least a portion of iodine in the iodine solution migrates into at least a portion of the conductive hydrogel (or conductive adhesive) prior to or during step (d) or (iv), whereby the conductive hydrogel/adhesive comprises iodine dispersed throughout at least a portion of the hydrogel.

Illustrative embodiment 5B. An iodine solution for use in the method of any of illustrative embodiments 1-5A.

Illustrative embodiment 6. A kit, comprising: a conductive hydrogel (or conductive adhesive) for application to a patient's skin and for placement between the patient's skin and at least one transducer array that generates an alternating electric field, wherein the conductive hydrogel (or conductive adhesive) comprises a first surface and a second surface; and an iodine solution for disposal on at least a portion of the patient's skin or for disposal on at least a portion of a surface (such as, but not limited to, the second surface) of the conductive hydrogel (or conductive adhesive).

Illustrative embodiment 7. The kit of claim 6, wherein the iodine solution is selected from the group consisting of povidone-iodine, Iodixanol, an iodophor, elemental iodine, complexed iodine, and combinations thereof; optionally, wherein the iodine solution includes an iodide salt (e.g., sodium iodide, silver iodide, etc.) or hydrogen iodide.

Illustrative embodiment 7A. The kit of Illustrative embodiment 6 or 7, wherein the iodine solution comprises an iodophor, and wherein the iodophor comprises iodine complexed with at least one solubilizing agent.

Illustrative embodiment 7B. The kit of Illustrative embodiment 7A, wherein the solubilizing agent is selected from the group consisting of povidone, polyvinylpyrrolidone, polyvinyl alcohol, starch derivatives, chitosan, cellulose, and combinations thereof.

Illustrative embodiment 8. The kit of any of Illustrative embodiments 6-7B, wherein the conductive hydrogel (or conductive adhesive) is a semi-solid conductive hydrogel (or conductive adhesive) having a thickness in a range of from about 25 μm to about 2500 μm, and/or a range of from about 1 mil to about 100 mil.

Illustrative embodiment 9. The kit of any one of Illustrative embodiments 6-8, further comprising at least one pair of transducer arrays that generates an alternating electric field having a frequency in a range of from about 50 kHz to about 1 MHz upon application to a patient's skin in combination with the conductive hydrogel (or conductive adhesive) and the iodine solution.

Illustrative embodiment 9A. The kit of any of illustrative embodiments 6-9, further comprising instructions for applying at least a portion of the iodine solution and the conductive hydrogel (or conductive adhesive) to a skin of a subject.

Illustrative embodiment 9B. The kit of any of illustrative embodiments 6-9A, further comprising at least one dermatological therapeutic agent.

Illustrative embodiment 9C. The kit of illustrative embodiment 9B, wherein the dermatological therapeutic agent is selected from the group consisting of an anti-microbial agent, an antibiotic, an anti-viral agent, an anti-fungal agent, an anti-inflammatory agent, an anesthetic, an emollient, a cleansing agent, an astringent, and combinations thereof.

Illustrative embodiment 9D. The kit of any of illustrative embodiments 6-9C, wherein the kit has a shelf life of at least about six months.

Illustrative embodiment 9E. The kit of any of illustrative embodiments 6-9D, further comprising a sealed packaging in which the conductive hydrogel (or conductive adhesive) and the iodine solution/coating are disposed, wherein the sealed packaging is substantially impermeable to air and/or substantially impermeable to light.

Illustrative embodiment 10. An assembly, comprising: a conductive hydrogel (or conductive adhesive) for placement between at least one electrode element and a patient's skin, the conductive hydrogel (or conductive adhesive) having a first surface and a second surface, wherein the first surface of the conductive hydrogel (or conductive adhesive) is designed and configured to adhere to at least a portion of a surface of the at least one electrode, and the second surface of the conductive hydrogel (or conductive adhesive) is for application to a patient's skin; and a coating comprising, or formed from, an iodine solution, wherein the coating is in contact with at least a portion of the second surface of the conductive hydrogel (or conductive adhesive).

Illustrative embodiment 11. The assembly of Illustrative embodiment 10, wherein the assembly lacks homogeneity of iodine migrating into the conductive hydrogel (or conductive adhesive) between the second surface and the first surface.

Illustrative embodiment 12. The assembly of Illustrative embodiment 10 or 11, wherein the iodine solution is selected from the group consisting of povidone-iodine, Iodixanol, an iodophor, elemental iodine, complexed iodine, and combinations thereof, optionally, wherein the iodine solution includes an iodide salt (e.g., sodium iodide, silver iodide, etc.) or hydrogen iodide.

Illustrative embodiment 12A. The assembly of any of Illustrative embodiments 10-12, wherein the iodine solution comprises an iodophor, and wherein the iodophor comprises iodine complexed with at least one solubilizing agent.

Illustrative embodiment 12B. The assembly of Illustrative embodiment 12A, wherein the solubilizing agent is selected from the group consisting of povidone, polyvinylpyrrolidone, polyvinyl alcohol, starch derivatives, chitosan, cellulose, and combinations thereof.

Illustrative embodiment 13. The assembly of any one of Illustrative embodiments 10-12B, wherein the conductive hydrogel (or conductive adhesive) is a semi-solid conductive hydrogel (or conductive adhesive) having a thickness in a range of from about 25 μm to about 2500 μm and/or a range of from about 1 mil to about 100 mil.

Illustrative embodiment 14. The assembly of any one of Illustrative embodiments 10-13, further comprising a liner disposed on the iodine solution or coating and covering at least a portion of the second surface of the conductive hydrogel (or conductive adhesive).

Illustrative embodiment 15. The assembly of any one of Illustrative embodiments 10-14, further comprising at least one electrode, wherein the first surface of the conductive hydrogel (or conductive adhesive) is adhered to at least a portion of the surface of the at least one electrode.

Illustrative embodiment 16. The assembly of illustrative embodiment 15, wherein the at least one electrode generates an alternating electric field having a frequency in a range from about 50 kHz to about 500 kHz.

Illustrative embodiment 16A. An assembly, comprising: at least one electrode; a conductive hydrogel (or conductive adhesive) for placement between the at least one electrode and a patient's skin, the conductive hydrogel (or conductive adhesive) having a first surface and a second surface, wherein the first surface of the conductive hydrogel (or conductive adhesive) is adhered to at least a portion of a surface of the at least one electrode and the second surface of the conductive hydrogel (or conductive adhesive) is for application to a patient's skin; a coating comprising, or formed from, an iodine solution, wherein the coating is in contact with at least a portion of the second surface of the conductive hydrogel (or conductive adhesive).

Illustrative embodiment 17. A transducer array for a TTField-generating system, comprising: at least one assembly of any of Illustrative embodiments 15-16A.

Illustrative embodiment 18. The transducer array of Illustrative embodiment 17, further defined as comprising a plurality of assemblies in a range of from about 3 assemblies to about 30 assemblies.

Illustrative embodiment 19. A method, comprising: applying the assembly of any of Illustrative embodiments 10-16A to a skin of a patient; applying at least one electrode to the first surface of the conductive hydrogel (or conductive adhesive); and generating an alternating electric field for a period of time.

Illustrative embodiment 20. The method of Illustrative embodiment 19, wherein at least one of: the alternating electric field is applied at a frequency in a range of from about 50 kHz to about 1 MHz; the alternating electric field has a field strength of at least about 1 V/cm in at least a portion of a target region of the subject; the alternating electric field is induced by an applied voltage of at least 50 V RMS; and the period of time that the alternating electric field is applied is at least about 50% of at least about a 24 consecutive hour time period.

Illustrative embodiment 21. The assembly of any of illustrative embodiments 10-16A for use in the method of illustrative embodiment 19 or 20.

Illustrative embodiment 22. Use of the assembly of any of illustrative embodiments 10-16A in the method of illustrative embodiment 19 or 20.

Illustrative embodiment 23. A method, comprising: applying two or more of the transducer arrays of Illustrative embodiment 17 or 18 to a skin of a patient; and generating an alternating electric field for a period of time.

Illustrative embodiment 24. The method of Illustrative embodiment 23, wherein at least one of: the alternating electric field is applied at a frequency in a range of from about 50 kHz to about 1 MHz; the alternating electric field has a field strength of at least about 1 V/cm in at least a portion of a target region of the subject; the alternating electric field is induced by an applied voltage of at least 50 V RMS; and the period of time that the alternating electric field is applied is at least about 50% of at least about a 24 consecutive hour time period.

Illustrative embodiment 25. The transducer array of illustrative embodiment 17 or 18 for use in the method of illustrative embodiment 23 or 24.

Illustrative embodiment 26. A system, comprising: an iodine solution or coating applied to a skin of a subject; a conductive hydrogel (or conductive adhesive) applied or adhered to the skin to which the iodine solution or coating has been applied, the conductive hydrogel (or conductive adhesive) having a first surface and a second surface, wherein the second surface of the conductive hydrogel (or conductive adhesive) is in contact with the coated skin; at least one electrode applied or adhered to the first surface of the conductive hydrogel (or conductive adhesive).

Illustrative embodiment 27. The system of Illustrative embodiment 26, wherein the system is produced by the method of any of Illustrative embodiments 1-5, 19-20, and 23-24.

Illustrative embodiment 28. The system of Illustrative embodiment 27, wherein the system is further defined as comprising or produced from the kit of any of Illustrative embodiments 6-9E.

Illustrative embodiment 29. The system of Illustrative embodiment 27, wherein the system is further defined as comprising or produced from the assembly of any of Illustrative embodiments 10-16A and 21.

Illustrative embodiment 30. The system of Illustrative embodiment 27, wherein the system is further defined as comprising the transducer array of any of Illustrative embodiments 17-18 and 25.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method, comprising the steps of (a)-(d) or the steps of (i)-(iv):
(a) applying or contacting an iodine solution to a portion of a skin of a patient;
(b) applying or contacting a conductive hydrogel or a conductive adhesive to at least a portion of the skin to which the iodine solution has been applied;
(c) applying or contacting at least one transducer array for a TTField-generating system to at least a portion of the skin to which the iodine solution and conductive hydrogel or conductive adhesive have been applied; and
(d) generating an alternating electric field for a period of time; OR
(i) applying or contacting an iodine solution to a portion of a skin of a patient;
(ii) including a conductive hydrogel or a conductive adhesive as a component of at least one transducer array for a TTField-generating system, or applying or contacting a conductive hydrogel or a conductive adhesive to at least a portion of the at least one transducer array for a TTField-generating system;
(iii applying or contacting the at least one transducer array for the TTField-generating system to at least a portion of the skin to which the iodine solution has been applied thereby contacting the at least one transducer array to the skin of the patient via the conductive hydrogel or conductive adhesive; and
(iv) generating an alternating electric field for a period of time.

2. The method of claim 1, wherein at least one of:
the alternating electric field is applied at a frequency in a range of from about 50 kHz to about 1 MHz;
the alternating electric field has a field strength of at least about 1 V/cm in at least a portion of a target region of the subject;
the alternating electric field is induced by an applied voltage of at least 50 V RMS; and
the period of time that the alternating electric field is applied is at least about 50% of at least about a 24 consecutive hour time period.

3. The method of claim 1, wherein the iodine solution is selected from the group consisting of povidone-iodine, Iodixanol, an iodophor, elemental iodine, complexed iodine, and combinations thereof, optionally, wherein the iodine solution includes an iodide salt or hydrogen iodide.

4. The method of claim 3, wherein the iodine solution comprises an iodophor, and wherein the iodophor comprises iodine complexed with at least one solubilizing agent, optionally wherein the solubilizing agent is selected from the group consisting of povidone, polyvinylpyrrolidone, polyvinyl alcohol, starch derivatives, chitosan, cellulose, and combinations thereof.

5. The method of claim 1, wherein the conductive hydrogel or conductive adhesive has a first surface and a second surface, wherein the first surface of the conductive hydrogel or conductive adhesive adheres to at least a portion of a surface of the transducer array, and the second surface of the conductive hydrogel or conductive adhesive is applied to the skin to which the iodine solution has been applied.

6. The method of claim 1, wherein the conductive hydrogel or conductive adhesive is a semi-solid conductive hydrogel or conductive adhesive having a thickness in a range of from about 25 μm to about 2500 μm.

7. A kit, comprising:
a conductive hydrogel or conductive adhesive for application to a patient's skin and for placement between the patient's skin and at least one transducer array that generates an alternating electric field; and
an iodine solution for disposal on at least a portion of the patient's skin or for disposal on at least a portion of a surface of the conductive hydrogel or conductive adhesive.

8. The kit of claim 7, wherein the iodine solution is selected from the group consisting of povidone-iodine, Iodixanol, an iodophor, elemental iodine, complexed iodine, and combinations thereof, optionally, wherein the iodine solution includes an iodide salt or hydrogen iodide.

9. The kit of claim 7, wherein the conductive hydrogel or conductive adhesive is a semi-solid conductive hydrogel or conductive adhesive having a thickness in a range of from about 25 μm to about 2500 μm.

10. The kit of claim 7, further comprising at least one pair of transducer arrays that generates an alternating electric field having a frequency in a range of from about 50 kHz to about 1 MHz upon application to a patient's skin in combination with the iodine solution and the conductive hydrogel or conductive adhesive.

11. An assembly, comprising:
a conductive hydrogel or conductive adhesive for placement between at least one electrode element and a patient's skin, the conductive hydrogel or conductive adhesive having a first surface and a second surface, wherein the first surface of the conductive hydrogel or conductive adhesive is configured to adhere to at least a portion of a surface of the at least one electrode and the second surface of the conductive hydrogel or conductive adhesive is for application to a patient's skin; and
a coating comprising, or formed from, an iodine solution, wherein the coating is in contact with at least a portion of the second surface of the conductive hydrogel or conductive adhesive.

12. The assembly of claim 11, wherein the assembly lacks homogeneity of iodine migrating into the conductive hydrogel or conductive adhesive between the second surface and the first surface.

13. The assembly of claim 11, wherein at least one of:
the iodine solution is selected from the group consisting of povidone-iodine, Iodixanol, an iodophor, elemental iodine, complexed iodine, and combinations thereof, optionally, wherein the iodine solution includes an iodide salt or hydrogen iodide; and/or
the conductive hydrogel or conductive adhesive is a semi-solid conductive hydrogel or conductive adhesive having a thickness in a range of from about 25 μm to about 2500 μm.

14. The assembly of claim 11, further comprising a liner disposed on the iodine solution or coating and covering at least a portion of the second surface of the conductive hydrogel or conductive adhesive.

15. The assembly of claim 11, further comprising at least one electrode, wherein the first surface of the conductive hydrogel or conductive adhesive is adhered to at least a portion of the surface of the at least one electrode.

16. A transducer array for a TTField-generating system, comprising:
at least one assembly of claim 15.

17. The transducer array of claim 16, further defined as comprising a plurality of assemblies in a range of from about 3 assemblies to about 30 assemblies.

18. A method, comprising:
applying the assembly of claim 11 to a skin of a patient;
applying at least one electrode to the first surface of the conductive hydrogel or conductive adhesive; and
generating an alternating electric field for a period of time.

19. The method of claim 18, wherein at least one of:
the alternating electric field is applied at a frequency in a range of from about 50 kHz to about 1 MHz;
the alternating electric field has a field strength of at least about 1 V/cm in at least a portion of a target region of the subject;
the alternating electric field is induced by an applied voltage of at least 50 V RMS; and
the period of time that the alternating electric field is applied is at least about 50% of at least about a 24 consecutive hour time period.

20. A method, comprising:
applying two or more of the transducer arrays of claim 16 to a skin of a patient; and
generating an alternating electric field for a period of time.
